(19) United States
(12) Patent Application Publication
(10) Pub. No.: US 2026/0325453 A1
(43) Pub. Date: Oct. 22, 2026

(54) Polydopamine-Coated Nanoparticles in Neurons for Absorption of Ultra-Weak UV Photon Emission to Reduce Downstream Neurotoxic Effects and Neuronal Inflammation in Neurodegenerative Conditions

(71) Applicants: Kamran Ansari, Tustin, CA (US); Nadia Ansari, Tustin, CA (US)

(72) Inventors: Kamran Ansari, Tustin, CA (US); Nadia Ansari, Tustin, CA (US)

(21) Appl. No.: 19/647,498

(22) Filed: Apr. 14, 2026

Related U.S. Application Data
(60) Provisional application No. 63/790,221, filed on Apr. 17, 2025.

(51) Int. Cl. A61K 9/50; A61K 33/00; A61K 33/242; A61K 33/26; A61K 47/68; A61K 47/69; A61P 25/28; A61P 37/00

(52) U.S. Cl. CPC A61K 9/5031; A61K 33/00; A61K 33/242; A61K 33/26; A61K 47/6865; A61K 47/6929; A61P 25/28; A61P 37/00

(57) ABSTRACT

Polydopamine-coated nanoparticles are presented as a novel composition for intracranial delivery, designed to absorb UV photons and mitigate neuroinflammation in brain tissue. These nanoparticles feature a core that includes materials such as silica, gold, iron oxide, or polymeric micelles and are encapsulated within a polydopamine shell. Additionally, biologically active agents can be incorporated to enhance neuroprotection. Methods are provided for treating neurodegenerative diseases (e.g., Parkinson's, Huntington's, ALS, Alzheimer's) or improving glioblastoma therapy by administering the polydopamine-coated nanoparticles directly to affected brain regions via intracerebral injection, intrathecal infusion, or convection-enhanced delivery. Furthermore, the polydopamine-coated nanoparticles hold potential for pharmaceutical applications in developing medicaments targeting neuroinflammation and UV photon absorption within the central nervous system.

### CROSS-REFERENCE

The present application relies on U.S. Patent Provisional Application No. 63/790,221, tilted “Polydopamine-Coated Nanoparticles in Neurons for Absorption of Ultra-Weak UV Photon Emission to Reduce Downstream Neurotoxic Effects and Neuronal Inflammation in Neurodegenerative Conditions” and filed on Apr. 17, 2025, for priority, which is herein incorporated by reference in its entirety.

### FIELD

The present specification is related generally to the field of nanomedicine, neurology, and neurodegenerative diseases. More specifically, the present specification pertains to polydopamine-coated nanoparticles designed for intracranial or intraneuronal delivery to absorb ultraviolet (UV) photons, mitigate neuroinflammation, and thereby ameliorate or slow progression of neurological conditions such as Parkinson's disease, Huntington's chorea, amyotrophic lateral sclerosis (ALS), Alzheimer's disease, and glioblastoma resistance to treatment.

### BACKGROUND

A growing body of research suggests that heightened neuroinflammation can be accompanied by ultra-weak photon emissions (UPE), potentially in the UV (ultra-violet) range. Additionally, these UV UPE can result in other neurotoxic effects. UV light stimulates the generation of superoxide anion and hydrogen peroxide, creating a cycle of heightened chronic neuroinflammation. There is an accompanying decrease in tryptophan fluorescence and an increase in fluorescence by tryptophan metabolites, consistent with stimulation of the kynurenine pathway. This same mechanism also occurs within the brain, and is associated with the cytokines, interferon gamma and TNF-alpha, which stimulate excess production of the enzymes, indoleamine 2,3-dioxygenase and tryptophan 2,3-dioxygenase. Increased cytokines levels result in greater release of biophotons from mitochondria in the brain. Toxic levels of kynurenines are associated with the development of many neurological diseases, including Parkinson's disease, Huntington's chorea, amyotrophic lateral sclerosis and Alzheimer's, as well as glioblastoma resistance to treatment.

Polydopamine (PDA), a synthetic analog of mussel adhesive proteins, is known for its broad-spectrum optical absorption, including in the UV range. PDA can dissipate absorbed light energy primarily as heat or via non-radiative decay, potentially reducing UV-induced damage and oxidative stress in tissues. Polydopamine coatings also exhibit biocompatibility, strong surface adhesion to diverse materials, and anti-inflammatory or reduced-immunogenic properties.

Considering the benefits of polydopamine (PDA) and similar compositions, the present specification discloses the design and use of nanoparticles, preferably polydopamine-coated nanoparticles, for targeted and local administration to inflamed or diseased neural tissue. The polydopamine-coated nanoparticles aim to reduce the phototoxic and oxidative burden that exacerbates neurodegeneration and tumor aggressiveness by absorbing or quenching UV photons.

### SUMMARY

The following embodiments and aspects thereof are described and illustrated in conjunction with systems, tools and methods, which are meant to be exemplary and illustrative, and not limiting in scope. The present application discloses numerous embodiments.

The present specification discloses a composition comprising: a nanoparticle core; and a polydopamine coating disposed on the nanoparticle core, wherein the polydopamine coating is configured to absorb photons within a wavelength range of 200-400 nm, and wherein the composition, when present in neural tissue, attenuates ultra-weak photon emission by at least 5%.

Optionally, when present in the neutral tissue, the composition attenuates ultra-weak photon emission by at least 25%.

Optionally, the polydopamine coating dissipates absorbed energy via non-radiative decay.

Optionally, the nanoparticle core comprises silica, gold, or iron oxide.

Optionally, the composition further comprises a targeting ligand.

Optionally, the composition is present at 0.1-5 mg/mL in tissue.

Optionally, the composition further comprises a chemotherapeutic agent.

The present specification also discloses a method of reducing neuroinflammation in a subject comprising administering to neural tissue a plurality of polydopamine-coated nanoparticles, wherein the polydopamine-coated nanoparticles are adapted to absorb ultra-weak photon emission within the neural tissue and wherein absorption of the ultra-weak photon emission reduces reactive oxygen species levels in the neural tissue.

Optionally, the reactive oxygen species levels are reduced by at least 20%.

Optionally, in the neural tissue, cytokine levels are reduced by at least 15%.

Optionally, a kynurenine-to-tryptophan ratio is reduced.

The present specification also discloses a nanoparticle comprising: a core having a diameter of 50-150 nm; and a polydopamine coating having a thickness of 5-50 nm, wherein the nanoparticle composition exhibits an absorption coefficient of at least 10⁴ M⁻¹ cm⁻¹ within a wavelength range of 220-350 nm.

Optionally, the polydopamine coating thickness is 10-20 nm.

Optionally, the nanoparticle composition exhibits at least 25% attenuation of UV photons.

The present specification also discloses a method of treating a neurodegenerative disease, comprising administering a plurality of functionalized polydopamine-coated nanoparticles to a target region of the brain, wherein the nanoparticles are configured to localize to inflamed neural tissue and attenuate ultra-weak photon emission within the target region.

Optionally, the neurodegenerative disease is Alzheimer's disease.

Optionally, administration is via convection-enhanced delivery.

Optionally, the polydopamine-coated nanoparticles are conjugated to an antibody targeting glioblastoma cells.

The present specification also discloses a method comprising detecting ultra-weak photon emission from neural tissue; administering polydopamine-coated nanoparticles based on the detected emission; and reducing the detected photon emission following administration.

Optionally, the method further comprises adjusting dosage of the polydopamine-coated nanoparticles based on the detected ultra-weak photon emission.

The present specification also discloses a composition comprising: a nanoparticle core comprising at least a first material; and a coating encapsulating the nanoparticle core, wherein the coating comprises polydopamine.

Optionally, the first material is at least one of silica, gold, iron oxide, and polymeric micelle. Optionally, the composition further comprises biologically active agents attached to or encapsulated within the coating to enhance neuroprotection or reduce neuroinflammation.

The specification also discloses a method of treating or alleviating at least one neurodegenerative disease comprising administering an effective amount of the composition to a patient's brain.

Optionally, said administration is through intracranial delivery.

Optionally, the method further comprises using the administration of said composition to absorb UV photons and reduce neuroinflammation in brain tissue.

Optionally, the at least one neurodegenerative disease is at least one of Parkinson's disease, Huntington's disease, ALS disease, Alzheimer's disease or glioblastoma,

Optionally, said administration is by at least one of intracerebral injection, intrathecal infusion, or convection-enhanced delivery.

The present specification also discloses the use of the polydopamine-coated nanoparticles in the manufacture of a medicament for the absorption of UV photons and reduction of neuronal inflammation in the central nervous system.

The aforementioned and other embodiments of the present specification shall be described in greater depth in the drawings and detailed description provided below.

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries (e.g. boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1 shows a block diagram representation of a polydopamine-coated nanoparticle, in accordance with some embodiments of the present specification;

FIG. 2 is a flowchart of a plurality of steps of a method for the manufacture of a plurality of polydopamine-coated nanoparticles, in accordance with some embodiments of the present specification;

FIG. 3 is a flowchart describing various steps of a method for reducing neuroinflammation within neural tissues of a patient using polydopamine-coated nanoparticles, in accordance with some embodiments of the present specification;

FIG. 4 is a flowchart describing various steps of a method for treating neurodegenerative diseases of a patient using polydopamine-coated nanoparticles, in accordance with some embodiments of the present specification;

FIG. 5 is a flowchart of a plurality of steps of a method for administration of a plurality of polydopamine-coated nanoparticles to disease-affected cells/areas/tissues, in accordance with some embodiments of the present specification; and

FIG. 6 is a flowchart describing various steps of a method for administration of polydopamine-coated nanoparticles, in accordance with some embodiments of the present specification.

### DETAILED DESCRIPTION

The present specification is directed towards a therapeutic system comprising a plurality of polydopamine-coated nanoparticles that: a) absorb UV photons—each of the plurality of nanoparticles feature a core (which may include a metal, polymer, or silica) and a polydopamine shell that exhibits a broad UV absorption spectrum, b) reduce neuroinflammation—by curtailing local photon-induced oxidative stress, the plurality of polydopamine-coated nanoparticles potentially lower proinflammatory cytokine release, protecting neurons from ROS damage, and c) improve treatment outcomes—In Parkinson's disease, Huntington's disease, ALS, Alzheimer's disease (AD), and glioblastoma, local UV quenching and reduced inflammatory stress can slow or mitigate disease progression, while potentially enhancing the efficacy of co-administered therapeutics.

The plurality of polydopamine-coated nanoparticles may be delivered intracranially, for example, via infusion into the brain parenchyma or cerebrospinal fluid (CSF), or via targeted approaches such as convection-enhanced delivery, with the intent to concentrate these particles in disease-affected areas.

Alzheimer's disease (AD) is characterized by the formation of amyloid beta (Aβ) plaques, tau aggregates, and chronic neuroinflammation. The brain produces Aβ plaques in an attempt to absorb or quench ultraviolet (UV) light generated by the heightened neuroinflammatory processes.

There is a well-established link between UV exposure as a result of chronic neuroinflammation/reactive oxygen species (ROS) and tryptophan metabolism along the kynurenine pathway. UV exposure results in the production of more ROS, leading to more UV exposure, pointing towards a contribution for the chronic neuroinflammation cycle. The resultant build-up of ROS and neurotoxic kynurenine metabolites—implicated in multiple neurodegenerative disorders—may generate or exacerbate damaging biophotonic emissions in inflamed tissues.

UV radiation, particularly UVA and UVB induces the formation of reactive oxygen species (ROS) in affected/inflamed cells or tissues. The production of UV-induced ROS further damages cellular components, leading to oxidative stress, DNA damage and chronic neuroinflammation.

Chronic neuroinflammation, caused by ROS, activates enzymes like indoleamine 2,3-dioxygenase (IDO-1) and tryptophan 2,3-dioxygenase (TDO). These enzymes catalyze the first step in tryptophan metabolism, shifting/diverting its degradation away from serotonin and melatonin production towards kynurenine pathway. Further, ROS and oxidative stress enhance the activity of IDO and TDO thereby increasing kynurenine production. This shifting/diverting of tryptophan metabolism is commonly seen in neuroinflammatory conditions like multiple sclerosis, Alzheimer's disease, and depression. Further, the UV exposure can influence system tryptophan metabolism, not just locally in the skin but also in the brain through immune system signaling.

Kynurenine and its neurotoxic downstream metabolites, such as 3-hydroxykynurenine (3-HK) and quinolinic acid (QA) contribute to neurotoxicity through excitotoxicity and further ROS production. Conversely, kynurenine acid (KYNA), another metabolite, has neuroprotective effects but it is often reduced under inflammatory conditions. An increase of kynurenine-to-tryptophan ratio is often associated with immunosuppression and neuroinflammatory disorders. Additionally, a dysregulated kynurenine pathway due to UV-induced ROS and inflammation may contribute in neurodegenerative and psychiatric disorders. Excessive quinolinic acid (QA) can lead to excitotoxicity, implicated in depression, schizophrenia, and neurodegeneration.

Since amyloid beta (Aβ) fibrils serves as an efficient UV absorbers (or sinks), the brain produces them to mitigate the detrimental effects of this UV-induced photo-oxidative stress. Further, tryptophan-derived metabolites, accelerate neurodegeneration in tandem with cytokine-driven inflammation and microtubule destabilization.

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

In the description and claims of the application, each of the words “comprise”, “include”, “have”, “contain”, and forms thereof, are not necessarily limited to members in a list with which the words may be associated. Thus, they are intended to be equivalent in meaning and be open-ended in that an item or items following any one of these words is not meant to be an exhaustive listing of such item or items, or meant to be limited to only the listed item or items. It should be noted herein that any feature or component described in association with a specific embodiment may be used and implemented with any other embodiment unless clearly indicated otherwise.

It must also be noted that as used herein and in the appended claims, the singular forms “a,” “an,” and “the” include plural references unless the context dictates otherwise. Although any systems and methods similar or equivalent to those described herein can be used in the practice or testing of embodiments of the present disclosure, the preferred, systems and methods are now described.

### Therapeutic System

In accordance with some embodiments, the present specification is directed towards a therapeutic system comprising a plurality of polydopamine-coated nanoparticles. In some embodiments, each of the plurality of polydopamine-coated nanoparticles has a core nanoparticle and a shell, layer or coating.

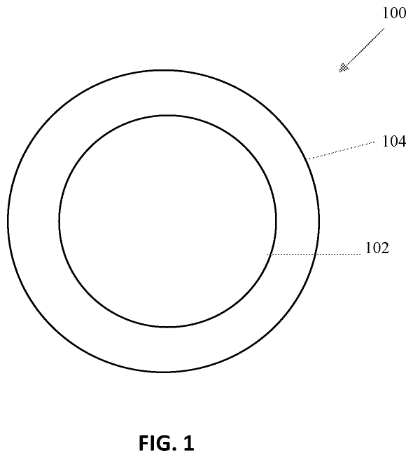

FIG. 1 shows a block diagram representation of a polydopamine-coated nanoparticle 100, in accordance with some embodiments of the present specification. The polydopamine-coated nanoparticle 100, being part of an engineered nanoscale system comprising a plurality of polydopamine-coated particles, is configured to physically interact with and attenuate naturally occurring photon emissions within biological tissues. In some embodiments, a plurality of polydopamine-coated nanoparticles 100 is configured to attenuate ultra-weak photon emission (UPE) within diseased neural tissue by at least 5%, at least 10%, at least 25%, at least 50%, or greater, as measure by photomultiplier-based detection systems or equivalent photon-counting instrumentation. The polydopamine-coated nanoparticles function as localized photon-attenuating structures with defined optical and structural properties, thereby transforming the microenvironment of diseased tissues. Each of the plurality of polydopamine-coated nanoparticles 100 comprises an inner core nanoparticle 102 and an outer coating, layer or shell 104.

### Core Nanoparticles 102

In some embodiments, a material for the core nanoparticles 102 can be, for example, silica, iron oxide, gold, polymeric micelles or liposomes. Different materials offer their own unique benefits to the core nanoparticles 102, for example (a) using silica as the material for the core nanoparticles 102 offers benefits such as, for example, a rigid matrix, easy functionalization and high biocompatibility, (b) using iron oxide as the material for the core nanoparticles 102 offers benefits such as, for example, MRI tracking and/or magnetically guided drug delivery, (c) using gold as the material for the core nanoparticles 102 offers benefits such as, for example, synergies with other therapeutic treatments, such as, but not limited to, photothermal therapy (PTT), and (d) using polymeric micelles or liposomes as the material for the core nanoparticles 102 offers benefits such as, for example, an ability to encapsulate additional drugs, such as, but not limited, to anti-inflammatory agents.

In some embodiments, a core diameter of each of the core nanoparticles 102 ranges from 50 nm (nanometers) to 200 nm (nanometers). It should be appreciated that the claimable range for the disclosed inventions could be any subset of the range from 50 nm (nanometers) to 200 nm (nanometers). The plurality of core nanoparticles 102 within this range maximize retention within neural tissues, provide optimal tissue penetration, and minimize clearance by microglia as well as reticuloendothelial system (RES), thereby prolonging photon absorption activity within inflamed regions.

### Method of Manufacturing the Core Nanoparticle 102

The procedure for the manufacture of the core nanoparticles 102 depends upon the material chosen for the core, for example, sol-gel synthesis is used for the manufacture of silica core nanoparticles 102, whereas, a seed-growth method is used for the manufacture of gold core nanoparticles 102.

### Shell, Layer or Coating 104

In some embodiments, each of the plurality of core nanoparticles 102 is coated with a layer of polydopamine (PDA) which exhibits UV (ultra-violet) and visible light absorption abilities. In some embodiments, the PDA coating 104 exhibits an absorption spectrum spanning at least 200 nm (nanometers) to 400 nm (nanometers), corresponding to ultraviolet wavelengths including UV-C, UV-B, and UV-A. In some embodiments, the PDA coating 104 further exhibits an absorption coefficient of at least 10⁴ M⁻¹ cm⁻¹ within one or more subranges of 220 nm (nanometers) to 350 nm (nanometers), thereby enabling effective attenuation of incident photon energy within the specified spectral range.

### Method of Manufacturing the Shell, Layer or Coating 104

In some embodiments, each of the plurality of core nanoparticles 102 is dispersed in a mildly alkaline solution containing dopamine concentrate which promotes oxidative polymerization of the dopamine monomers when stirred continuously for 6-24 hours at room temperature. This process leads to the formation of a uniform polydopamine (PDA) shell, layer or coating 104, which spontaneously deposits as a thin, adherent layer on the surface of each of the plurality of core nanoparticles 102. Thereafter, each of the plurality of core nanoparticles 102 is washed to remove any unreactive or loosely bound polymer or residual dopamine.

In embodiments, the thickness of the PDA coating 104, on each of the plurality of core nanoparticle, depends on a plurality of factors, such as, for example, reaction time and dopamine concentration. Longer reaction times lead to thicker coatings on the other hand, higher dopamine concentration leads to more deposition of the PDA, resulting in thicker coating. In some embodiments, the thickness of the PDA coating 104 is selected to optimize photon absorption efficiency while maintaining biocompatibility and tissue penetration. In some embodiments, the thickness of the PDA coating 104 on each of the plurality of core nanoparticles 102 ranges from 5 nm (nanometers) to 50 nm (nanometers) or more. A PDA coating 104 thickness within this range provides at least 20% greater photon attenuation relative to the plurality of core nanoparticles 102 having PDA coating 104 of thickness less than 5 nm (nanometers). In some embodiments, a ratio of core diameter to the thickness of PDA coating 104 is selected for each of the plurality of polydopamine-coated nanoparticles 100 to achieve a target photon attenuation efficiency of at least 25% within a defined wavelength range.

In some embodiments, the basicity/pH value of the alkaline dopamine solution is ~8.5. In some embodiments, the dopamine concentration in the mildly alkaline dopamine solution ranges from 0.1-2 mg/mL with a pH ranging from 8 to 9.

Once the PDA layer or shell is coated, each of the plurality of polydopamine-coated nanoparticles 100 is sterilized to ensure that they are free from contaminants. In some embodiments, processes such as filtration (with a filter having mesh size of about 0.22 μm) or gamma irradiation are preferred for sterilization depending upon the stability of the core material used. After sterilization, each of the plurality of polydopamine-coated nanoparticles 100 is resuspended in a sterile, isotonic, buffered solution to maintain physiological compatibility pH. In some embodiments, the sterile, isotonic, buffered solution is phosphate-buffered saline (PBS) at physiologically compatible pH.

### Optional Functionalization

In some embodiments, the surface of the polydopamine shell, layer or coating 104 can be further functionalized with peptides, antibodies, or aptamers targeting specific neurons, glial cells, or tumor cells (e.g., for glioblastoma). In some embodiments, the plurality of polydopamine-coated nanoparticles 100 is functionalized with one or more targeting moieties configured to preferentially localize to inflamed or diseased neural tissue. In certain embodiments, the targeting moieties comprise antibodies, peptides, or ligands having specificity for activated microglia, astrocytes, or tumor cells. In some embodiments, such targeted localization results in a local nanoparticle concentration of at least 0.1 mg/mL to 5 mg/mL within the target tissue, thereby enabling localized attenuation of ultra-weak photon emission.

In some embodiments, by leveraging covalent coupling, reactive groups (amine, catechol) of the polydopamine shell, layer or coating 104 can bind biologically active molecules, including antioxidants, growth factors, or chemotherapeutics.

### A Method of Manufacturing the Plurality of Polydopamine-Coated Nanoparticles 100

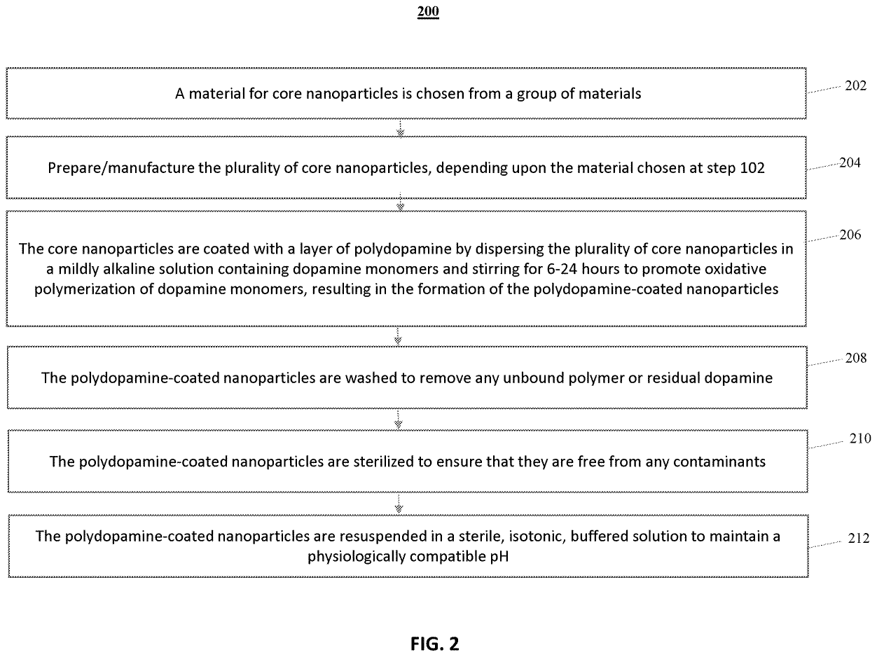

FIG. 2 is a flowchart of a plurality of steps of a method 200 for the manufacture of a plurality of polydopamine-coated nanoparticles 100, in accordance with some embodiments of the present specification.

Referring to FIGS. 1 and 2, at step 202, a material, for a plurality of core nanoparticles 102, is chosen from a group of materials. In various embodiments, the group of materials include silica, iron oxide, gold, polymeric micelles, or liposomes.

At step 204, a method is used for the preparation/manufacture of the plurality of core nanoparticles 102, depending upon the material chosen at step 102. For example, sol-gel synthesis is used for the manufacture of silica core nanoparticles 102 and, on the other hand, seed-growth method is used for the manufacture of gold core nanoparticles 102.

At step 206, the plurality of core nanoparticles 102 are coated with a layer of polydopamine by dispersing the plurality of core nanoparticles 102 in a mildly alkaline solution containing dopamine monomers and stirring for 6-24 hours to promote oxidative polymerization of dopamine monomers. This leads to the formation of a uniform polydopamine (PDA) shell, layer or coating 104, which spontaneously deposits as a thin, adherent layer on the surface of each of the plurality of core nanoparticles 102, thereby forming the plurality of polydopamine-coated nanoparticles 100.

In some embodiments, the basicity/pH value of the alkaline solution ranges from 8-9, on the other hand, the range of dopamine concentrate in the alkaline solution is 0.1 to 2 mg/mL.

At step 208, the plurality of polydopamine-coated nanoparticles 100 are washed to remove any unbound polymer or residual dopamine.

At step 210, the plurality of polydopamine-coated nanoparticles 100 are sterilized to ensure that they are free from any contaminants. In embodiments, processes such as filtration (with a filter having mesh size of about 0.22 μm) or gamma irradiation are preferred for sterilization depending upon the stability of the core material used.

At step 212, the plurality of polydopamine-coated nanoparticles 100 are resuspended in a sterile, isotonic, buffered solution to maintain a physiologically compatible pH. In some embodiments, the sterile, isotonic, buffered solution is phosphate-buffered saline (PBS) at physiologically compatible pH.

### Methods of Administering the Plurality of Polydopamine-Coated Nanoparticles 100

In some embodiments, the plurality of polydopamine-coated nanoparticles 100 can be delivered using methods such as, for example, intracerebral injections, intrathecal delivery or convection-enhanced delivery (CED).

To deliver an intracerebral injection, small burr holes or stereotactic guidance is used to deliver the plurality of polydopamine-coated nanoparticles 100 in proximity to the affected areas of the brain. In some embodiments, the intracerebral injection is used to deliver the plurality of polydopamine-coated nanoparticles 100 to substantia nigra, an affected area of the brain, of an individual suffering from Parkinson's disease. In some embodiments, the intracerebral injection is used to deliver the plurality of polydopamine-coated nanoparticles 100 to hippocampus, an affected area of the brain, of an individual suffering from Alzheimer's disease.

For intrathecal delivery, the plurality of polydopamine-coated nanoparticles 100 are injected or infused into the subarachnoid space. The flow of the cerebrospinal fluid (CSF) in the subarachnoid space helps in distributing the plurality of polydopamine-coated nanoparticles 100 throughout the brain and spinal cord.

For convection enhanced delivery (CED), the plurality of polydopamine-coated nanoparticles 100 are forced or driven deep into intraparenchymal tissue by using positive pressure. This method is commonly used in glioblastoma therapy to overcome the blood-brain barrier and reach deep seated tumor cells.

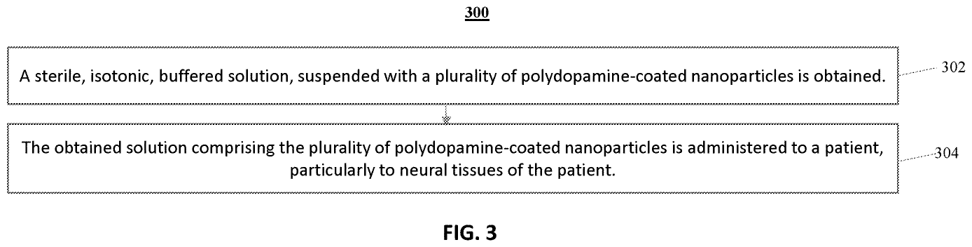

FIG. 3 is a flowchart describing various steps of a method 300 for reducing neuroinflammation within neural tissues of a patient using plurality of polydopamine-coated nanoparticles 100, in accordance with some embodiments of the present specification. Referring to FIGS. 1 and 3, at step 302, a sterile, isotonic, buffered solution, suspended with polydopamine-coated nanoparticles 100 is obtained.

At step 302, the obtained solution comprising the polydopamine-coated nanoparticles 100 is administered to neural tissues of a patient. Neural tissue, in a broad sense, refers to any specialized biological tissue composed of electrically excitable cells and their supporting elements that are organized to generate, transmit, process, or modulate electrochemical signals within an organism. It encompasses both the primary signaling cells (neurons) and associated support cells (glial cells), along with their extracellular environment, and is characterized by its role in communication, coordination, and regulation of physiological functions. Anatomical examples of neural tissue include the gray and white matter of the brain, the spinal cord, peripheral nerves such as the sciatic nerve, cranial nerves like the optic nerve, sensory ganglia including the dorsal root ganglion, autonomic ganglia such as the sympathetic chain ganglia, and specialized neural structures like the retina, all of which contain organized networks of neurons and glia adapted for specific signaling functions.

On administration, the plurality of polydopamine-coated nanoparticles 100, within the obtained solution, absorbs ultra-weak photon emissions within the administered neural tissues, resulting in reduced levels of reactive of oxygen species (ROS) compared to untreated or unadministered areas. In various embodiments, reactive oxygen species (ROS) levels are reduced by at least 20%.

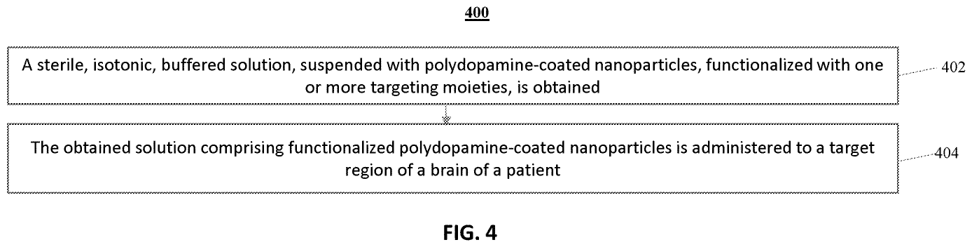

FIG. 4 is a flowchart describing various steps of a method 400 for treating neurodegenerative diseases of a patient using plurality of polydopamine-coated nanoparticles 100, in accordance with some embodiments of the present specification.

Referring to FIGS. 1 and 4, at step 402, a sterile, isotonic, buffered solution, suspended with a plurality of polydopamine-coated nanoparticles 100, functionalized with one or more targeting moieties, is obtained. In some embodiments, the targeting moieties comprise antibodies, peptides, or ligands having specificity for activated microglia, astrocytes, or tumor cells.

At step 402, the obtained solution comprising functionalized plurality of polydopamine-coated nanoparticles 100 is administered to a target region of a brain of a patient. On administration, the functionalized plurality of polydopamine-coated nanoparticles 100, within the obtained solution, localize to inflamed neural tissues and attenuate ultra-weak photon emission within the target region.

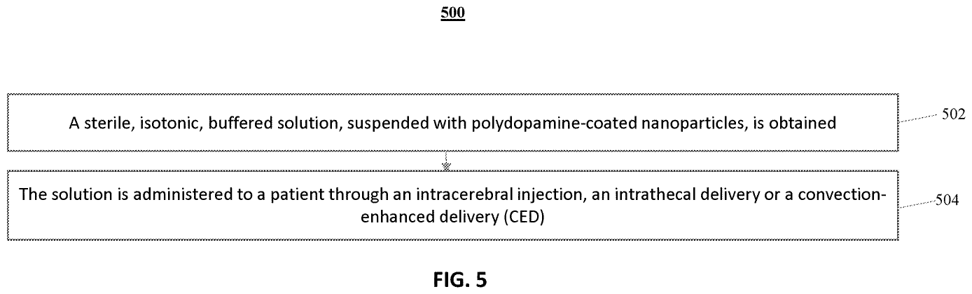

FIG. 5 is a flowchart of a plurality of steps of a method 500 for administration of a plurality of polydopamine-coated nanoparticles to disease-affected cells/areas/tissues, in accordance with some embodiments of the present specification.

Referring to FIGS. 1 and 5, at step 502, a sterile, isotonic, buffered solution, suspended with a plurality of polydopamine-coated nanoparticles 100 is obtained.

At step 504, the obtained solution is administered to a patient through an intracerebral injection, an intrathecal delivery or a convection-enhanced delivery (CED).

The intracerebral injection delivers the solution containing the plurality of polydopamine-coated nanoparticles 100 in proximity to the affected areas of a patient's brain using small burr holes or stereotactic guidance.

For intrathecal delivery, the solution containing the plurality of polydopamine-coated nanoparticles 100 is injected or infused into subarachnoid space. The flow of the cerebrospinal fluid (CSF) in the subarachnoid space helps in distributing the plurality of polydopamine-coated nanoparticles 100 throughout the brain and the spinal cord.

For convection-enhanced delivery, the solution containing the plurality of polydopamine-coated nanoparticles 100 is injected or infused into subarachnoid space. The flow of the cerebrospinal fluid (CSF) in the subarachnoid space helps in distributing the plurality of polydopamine-coated nanoparticles 100 throughout the brain and the spinal cord.

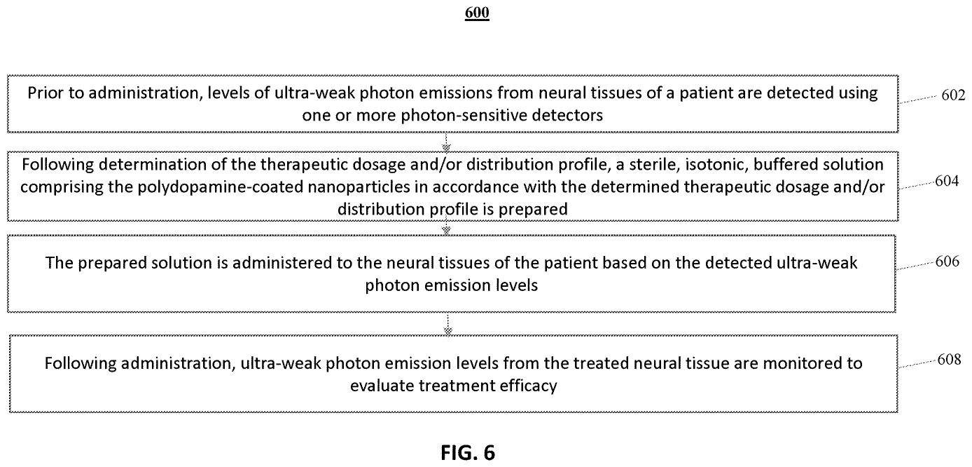

FIG. 6 is a flowchart describing various steps of a method 600 for administration of a plurality of polydopamine-coated nanoparticles 100, in accordance with some embodiments of the present specification.

Referring to FIGS. 1 and 6, at step 602, prior to administration, levels of ultra-weak photon emissions from neural tissues of a patient are detected using one or more photon-sensitive detectors. Post detection, the detected levels of ultra-weak photon emissions are used to determine a therapeutic dosage and/or a distribution profile of plurality of polydopamine-coated nanoparticles 100.

At step 604, following determination of the therapeutic dosage and/or distribution profile, a sterile, isotonic, buffered solution comprising the plurality of polydopamine-coated nanoparticles 100 in accordance with the determined therapeutic dosage and/or distribution profile is prepared.

At step 606, the prepared solution is administered to the neural tissues of the patient based on the detected ultra-weak photon emission levels. On administration, the plurality of polydopamine-coated nanoparticles 100 in accordance with the determined therapeutic dosage and/or distribution profile, within the prepared solution, reduces the detected ultra-weak photon emission levels.

At step 608, following administration, ultra-weak photon emission levels from the treated neural tissue are monitored to evaluate treatment efficacy. In some embodiments, the monitored photon emission levels are used to quantify reductions in photon flux relative to pre-administration levels. Based on the measured reduction in photon flux, subsequent dosing of the plurality of polydopamine-coated nanoparticles is adjusted to achieve a desired therapeutic outcome.

### Mechanisms of Action

The plurality of polydopamine-coated nanoparticles 100 slow or mitigate progression of neurodegenerative diseases, such as but not limited to Parkinson's disease, Huntington's disease, amyotrophic lateral sclerosis (ALS), Alzheimer's disease and glioblastoma, by reducing neuroinflammation of affected/inflamed cells/tissues/areas by exhibiting broad UV quenching or UV absorption ability, by potentially lowering proinflammatory cytokine release as well as by curtailing local photon-induced oxidative stress thereby protecting neurons from ROS damage.

With respect to UV quenching, elevated neuroinflammation (such as in case of neurodegenerative diseases) correlates with increased ultra-weak photon emissions (UPE) particularly in the UV spectrum. The plurality of polydopamine-coated nanoparticles 100 is delivered to the inflamed and affected cells, tissues or areas so that catechol and indole-like structures present in the polydopamine coating 104 of each of the plurality of polydopamine-coated nanoparticles 100 absorb the UV and visible light photons and dissipates the absorbed photon light energy through very small amounts of heat or via non-radiative decay pathways, such as for example, vibrational relaxation, resulting in localized thermal dissipation of less than 1° C. temperature increase in surrounding tissues. In some embodiments, attenuation of UV photon flux by the plurality of polydopamine coated nanoparticles results in a measurable reduction in ROS production in affected/inflamed cells/tissues/areas, such as for example, neuronal or glial cells.

Reducing oxidative damage, in-turn, reduces local phototoxic stress, potentially preventing damage associated with excessive UV exposure at cellular level. With fewer high-energy photons, there is less production of reactive oxygen species (ROS), less lipid peroxidation, and potentially diminished cytokine release by astrocytes and microglia. In some embodiments, ROS levels are reduced by at least 5%, at least 10%, or at least 15%. In certain embodiments, ROS levels are reduced by at least 25%, and in other embodiments by at least 50%. In further embodiments, the reduction in ROS levels ranges from about 5% to about 50% relative to untreated controls, as measured using fluorescence-based ROS assays. In some embodiments, the reduction in photon-induced oxidative stress results in decreased expression of pro-inflammatory cytokines, including TNF-α and IL-1β, by at least 10-40%, as measured by ELISA or equivalent assays. The polydopamine coating 104 on each of the plurality of polydopamine-coated nanoparticles 100 itself has intrinsic anti-inflammatory properties thereby it may help in minimizing overactivation of microglia, further lowering ROS levels and dampening local immune responses in the brain.

Neuroprotective and anti-tumor support-Since oxidative stress contributes to neurodegeneration, reducing UV induced oxidative damage may protect vulnerable neurons from further damage, slow disease progression by preserving neuronal function and may also improve functional outcomes, potentially benefiting cognitive and motor functions. In some embodiments, modulation of photon flux alters tryptophan metabolism, resulting in a decrease in the kynurenine-to-tryptophan ratio by at least 10%, thereby mitigating neurotoxic metabolite accumulation.

In some embodiments, optional functionalization of the surface of polydopamine coating 104 of each of the polydopamine-coated nanoparticles 100 with chemotherapeutics (for example temozolomide) may increase tumor cell kill while reducing resistance mechanisms.

### Efficacy in Disease Models

The effects of polydopamine-coated nanoparticles 100 are testable in both in-vitro as well as in-vivo model case studies.

### In-Vitro Studies

In these studies, neuron-astrocyte co-culture, a lab-based system where neurons and astrocytes are grown together, may be subjected to conditions mimicking oxidative stress or inflammation, such as due to UV irradiation (wavelength range 280-320 nm, intensity 0.5-2 μW/cm²) or inflammatory molecules (e.g., cytokines). On exposure of the cells with a 1 mg/ml plurality of polydopamine-coated nanoparticles 100 (100 nm diameter with 10 nm coating thickness), the neuron-astrocyte co-culture may show reduced levels of ROS, approximately 32% reduction in ROS levels (p<0.01), compared to untreated controls, resulting in improved cell survival and lower pro-inflammatory markers (like for example IL-β and TNF-α).

### In-Vivo Studies

In an in-vivo case study, a transgenic AD (Alzheimer's disease) mouse, when repeatedly treated (weekly for 4 weeks) with a 50 μL of a 1 mg/mL suspension of a plurality of polydopamine-coated nanoparticles 100, delivered via intracerebral administration (intraventricular injections), may show a reduction in beta-amyloid plaque load (p<0.05) in a range of at least 5%, preferably any amount in a range of 5% to 70% and may show reductions in microglial markers in the hippocampus of the brain in a range of at least 5%, preferably any amount in a range of 5% to 70%.

In a second in-vivo case study glioblastoma xenografts, when treated with the plurality of polydopamine-coated nanoparticles 100, delivered via intraparenchymal injections, may show slowed tumor growth and improved sensitivity to chemotherapeutic agents relative to tumors not treated with polydopamine-coated nanoparticles.

### Exemplary Use Cases

### First Use Case: Silica-PDA Nanoparticles for AD

A 50 μL injection of a 1 mg/mL nanoparticles suspension, prepared by coating each of 100 nm silica core nanoparticles with a ~10 nm of polydopamine shell, is administered into the hippocampus of a transgenic AD (Alzheimer's disease) mouse. Post administration of the injection, there is: (a) 30% reduction in load versus control group, (b) lower levels of IL-1β and TNF-α detectable in peri-plaque regions and (c) improved performance on Morris water maze, a test used to measure spatial learning and memory, indicating improved cognitive function.

### Second Use Case: Gold-PDA Nanoparticles in Glioblastoma

70 nm gold core nanoparticles each coated with ~15 nm of polydopamine shell, are coupled with anti-EGFR antibodies for targeted tumor binding and administered via convection-enhanced delivery (CED) to a tumor site in rat glioblastoma model. Post administration, there is: (a) enhanced chemotherapeutic penetration leading to a ~25% decrease/reduction in tumor volume and (b) fewer/reduced oxidative-stress markers (8-OHdG) in the surrounding tissue, indicating potential neuroprotection and reduced damage from reactive oxygen species (ROS).

### Safety and Toxicity Considerations

Although, the polydopamine-coated nanoparticles 100, of the present specification, are safe to be used in biological applications, careful control over their dose, degradation, and size is necessary to ensure that the polydopamine-coated nanoparticles 100 function effectively without causing any harm.

### Dose

The polydopamine coating 104 on each of the plurality of polydopamine-coated nanoparticles 100 is generally well-tolerated, that is, the body does not strongly react against the polydopamine coating 104. However, the dose must be optimized to prevent nanoparticle aggregation or prolonged microglial activation.

### Long-Term Degradation

The polydopamine coating 104 on each of the plurality of polydopamine-coated nanoparticles 100 is relatively stable, however, partial biodegradation may occur via oxidative processes in vivo, but the degradation byproducts are non-toxic.

### Size

The polydopamine-coated nanoparticles 100 in the central nervous system (CNS) may be cleared gradually. Therefore, a size range of 50-200 nm (preferably 50-100 nm), of each of the plurality of polydopamine-coated nanoparticles 100 is an optimal size range that improves potential lymphatic or microglial uptake and for clearance without causing severe cytotoxicity.

Thus, the present specification provides a novel and effective approach for mitigating the damaging effects of UV-induced oxidative stress in neurodegenerative and oncologic contexts. By coating nanoparticles with polydopamine, UV photons can be absorbed or “quenched”, reducing downstream inflammatory signaling and tissue damage. This strategy holds promise for reducing plaque formation in Alzheimer's disease, protecting dopaminergic neurons in Parkinson's disease, slowing progression in Huntington's disease and ALS, and overcoming some treatment resistance in glioblastoma.

The above examples are merely illustrative of the many applications of the therapeutic system of present specification. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

What is claimed is:

1. A composition comprising:
a nanoparticle core; and
a polydopamine coating disposed on the nanoparticle core, wherein the polydopamine coating is configured to absorb photons within a wavelength range of 200-400 nm, and wherein the composition, when present in neural tissue, attenuates ultra-weak photon emission by at least 5%.

2. The composition of claim 1, wherein, when present in the neutral tissue, the composition attenuates ultra-weak photon emission by at least 25%.

3. The composition of claim 1, wherein, the polydopamine coating dissipates absorbed energy via non-radiative decay.

4. The composition of claim 1, wherein the nanoparticle core comprises silica, gold, or iron oxide.

5. The composition of claim 1, further comprising a targeting ligand.

6. The composition of claim 1, wherein the composition is present at 0.1-5 mg/mL in tissue.

7. The composition of claim 1, further comprising a chemotherapeutic agent.

8. A method of reducing neuroinflammation in a subject comprising:
administering to neural tissue a plurality of polydopamine-coated nanoparticles, wherein the polydopamine-coated nanoparticles are adapted to absorb ultra-weak photon emission within the neural tissue and wherein absorption of the ultra-weak photon emission reduces reactive oxygen species levels in the neural tissue.

9. The method of claim 8, wherein the reactive oxygen species levels are reduced by at least 20%.

10. The method of claim 8, wherein, in the neural tissue, cytokine levels are reduced by at least 15%.

11. The method of claim 8, wherein kynurenine-to-tryptophan ratio is reduced.

12. A nanoparticle comprising:
a core having a diameter of 50-150 nm; and
a polydopamine coating having a thickness of 5-50 nm, wherein the nanoparticle composition exhibits an absorption coefficient of at least 10⁴ M⁻¹ cm⁻¹ within a wavelength range of 220-350 nm.

13. The nanoparticle composition of claim 12, wherein the polydopamine coating thickness is 10-20 nm.

14. The nanoparticle composition of claim 12, wherein the nanoparticle composition exhibits at least 25% attenuation of UV photons.

15. A method of treating a neurodegenerative disease, comprising:
administering a plurality of functionalized polydopamine-coated nanoparticles to a target region of the brain, wherein the nanoparticles are configured to localize to inflamed neural tissue and attenuate ultra-weak photon emission within the target region.

16. The method of claim 15, wherein the neurodegenerative disease is Alzheimer's disease.

17. The method of claim 15, wherein administration is via convection-enhanced delivery.

18. The method of claim 15, wherein the polydopamine-coated nanoparticles are conjugated to an antibody targeting glioblastoma cells.

19. A method comprising:
detecting ultra-weak photon emission from neural tissue;
administering polydopamine-coated nanoparticles based on the detected emission; and
reducing the detected photon emission following administration.

20. The method of claim 19, further comprising adjusting dosage of the polydopamine-coated nanoparticles based on the detected ultra-weak photon emission.
